(19) United States
(12) Patent Application Publication
(10) Pub. No.: US 2025/0176890 A1
(43) Pub. Date: Jun. 5, 2025

(54) ECG ELECTRODE WITH RETENTION FEATURE

(71) Applicant: KPR U.S., LLC, Dublin, OH (US)

(72) Inventors: Kevin BEAULIEU, Belchertown, MA (US); Erick GARSTKA, Westfield, MA (US); Michael CHU, Springfield, MA (US); Garrett LAWLOR, Framingham, MA (US)

(21) Appl. No.: 18/962,578

(22) Filed: Nov. 27, 2024

Related U.S. Application Data
(60) Provisional application No. 63/605,431, filed on Dec. 1, 2023.

(51) Int. Cl. A61B 5/28

(52) U.S. Cl. CPC A61B 5/28

(57) ABSTRACT

A biomedical electrode, and in particular an electrocardiogram (ECG) electrode, has one or more retention features for retaining a connector of a lead wire on the electrode. A retention member of a stud of the electrode includes a first retainer for engaging and retaining a first type of connector to the stud and a second retainer for engaging and retaining a second type of connector to the stud, where the first and second types of connectors having different connection methods.

### CROSS-REFERENCE TO RELATED APPLICATION

The present application claims the benefit of U.S. Provisional Application Ser. No. 63/605,431, filed Dec. 1, 2023, the entirety of which is hereby incorporated by reference.

### FIELD

The present disclosure generally relates to biomedical electrodes, and in particular, to an electrocardiogram (ECG) electrode placed on a patient's body and configured to attach a lead wire to connect the electrode to a monitor, the electrode having one or more retention features for retaining a connector of the lead wire on the electrode.

### BACKGROUND

When a patient requires monitoring for observation, treatment, or a combination of both, such as in a medical environment, e.g., a hospital, nursing home, or assisted living facility, the patient's vital signs and other health indicators may be monitored in order to continually and accurately assess the patient's well-being. One such vital sign is the monitoring of the heart via an electrocardiogram, which may be commonly referred to as an EKG and/or ECG.

Electrocardiogramonitors are widely used to obtain medical (i.e. biopotential) signals containing information indicative of the electrical activity associated with the heart and pulmonary system. To obtain medical signals, ECG electrodes are applied to the skin of a patient in various locations. The electrodes, after being positioned on the patient, connect to an ECG monitor by a set of ECG lead wires. The distal end of the ECG lead wire, or portion closest to the patient, may include a connector which is adapted to operably connect to the electrode to receive medical signals from the body. The proximal end of the ECG lead set is operably coupled to the ECG monitor and supplies the medical signals received from the body to the ECG monitor.

To monitor events of the heart via an ECG, a series of 3, 5, 6, 10, or 14 or more electrodes may be placed on a patient to sense electrical signals corresponding to activity of a patient's heart. For example, each of the electrodes may be used to allow the charge carriers (electrons) within the electrodes to communicate with the charge carriers (ions) within the body via electrochemical exchange. ECG electrodes on the body surface of a patient allows for voltage changes within the body to be recorded and/or displayed to a heath professional after adequate amplification of the signal.

### SUMMARY

In one aspect, an electrode assembly generally comprises electrode pad including a patient contact side for engaging skin of a patient and a connector side for attaching a connector to the electrode pad. A stud is mounted on the connector side of the electrode pad for directly engaging the connector to retain the connector to the electrode pad. The stud comprises a first retainer for engaging and retaining a first type of connector to the stud and a second retainer for engaging and retaining a second type of connector to the stud. The first and second types of connectors have different connection methods.

In another aspect, a stud for use in an electrode assembly generally comprises a projection configured for directly engaging a connector to retain the connector to the stud. The projection comprises a first retainer for engaging and retaining a first type of connector to the stud and a second retainer for engaging and retaining a second type of connector to the stud. The first and second types of connectors have different connection methods.

In yet another aspect, an electrode assembly generally comprises an electrode pad including a patient contact side for engaging skin of a patient and a connector side for attaching a snap-type connector to the electrode pad. A stud is mounted on the connector side of the electrode pad for directly engaging the snap-type connector to retain the snap-type connector to the electrode pad. The stud comprises a retainer for engaging and retaining the snap-type connector to the stud. A removal force of at least 2 lbs (8.90 N) is required for detaching the snap-type connector from the electrode assembly.

### BRIEF DESCRIPTION OF THE DRAWINGS

FIG. 1 is an illustration of a patient with a plurality of electrode assemblies attached to the patient and a lead set connected to the electrode assemblies;

FIG. 2 is an exploded view of an electrode assembly of the present disclosure;

FIG. 3 is an exploded view of a stud and eyelet assembly of the electrode assembly;

FIG. 4 is a perspective of the stud;

FIG. 5 is a side view of the stud;

FIG. 6 is a top view of the stud;

FIG. 7 is a perspective of a connector of the lead set;

FIG. 8 is a bottom view of the connector;

FIG. 9 is a perspective of a stud of another embodiment;

FIG. 10 is a side view of the stud in FIG. 9;

FIG. 11 is a top view of the stud in FIG. 9;

FIG. 12 is a perspective of a stud of another embodiment;

FIG. 13 is a side vide of the stud in FIG. 12; and

FIG. 14 is an illustration of a testing apparatus performing a retention force test on electrode assembly including the stud in FIG. 12.

Corresponding reference characters indicate corresponding parts throughout the drawings.

### DETAILED DESCRIPTION

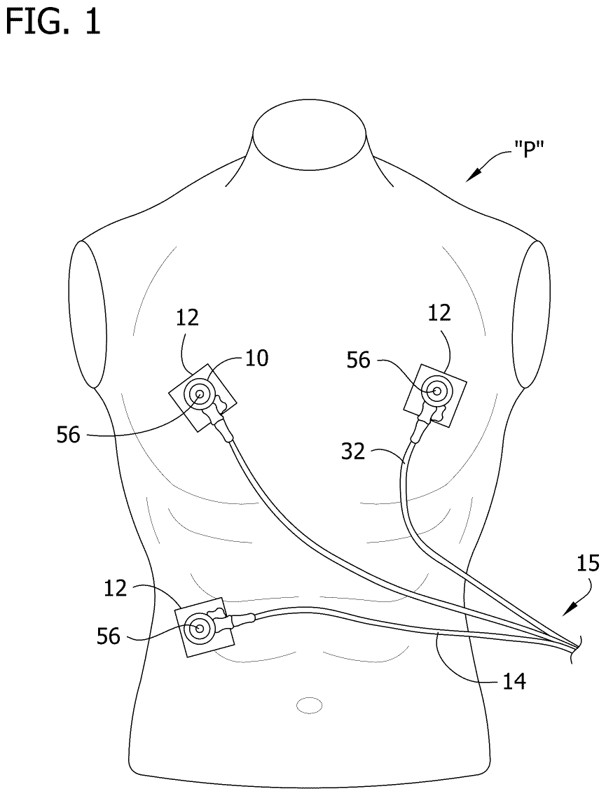

One or more aspects of the present disclosure pertain to biomedical electrodes that may be attached to a patient's skin in order to measure (or monitor) the electrical activity of the patient's heart and transmit a signal to a monitoring device, in addition to methods of use thereof. Referring to FIG. 1, a plurality of electrode assemblies 12 may be placed at various locations on a patient's body to monitor electrical activity of the heart and electrode connectors 10 may be connected to the electrodes. Movement by the patient can cause forces to be applied to the electrode connectors 10 and lead wires 14 extending from the connectors. Such motion may result in one or more of the connectors 10 being detached from the electrode assemblies 12. However, by providing the electrode features as described herein, the electrode assemblies 12 may provide for a more secure attachment with the connectors 10 to prevent inadvertent detachment of the connectors 10. Additionally, one or more features of the electrode assemblies 12 described herein provide for universal attachment to a variety of different types of connectors 10. Still other aspects of the disclosure are discussed below.

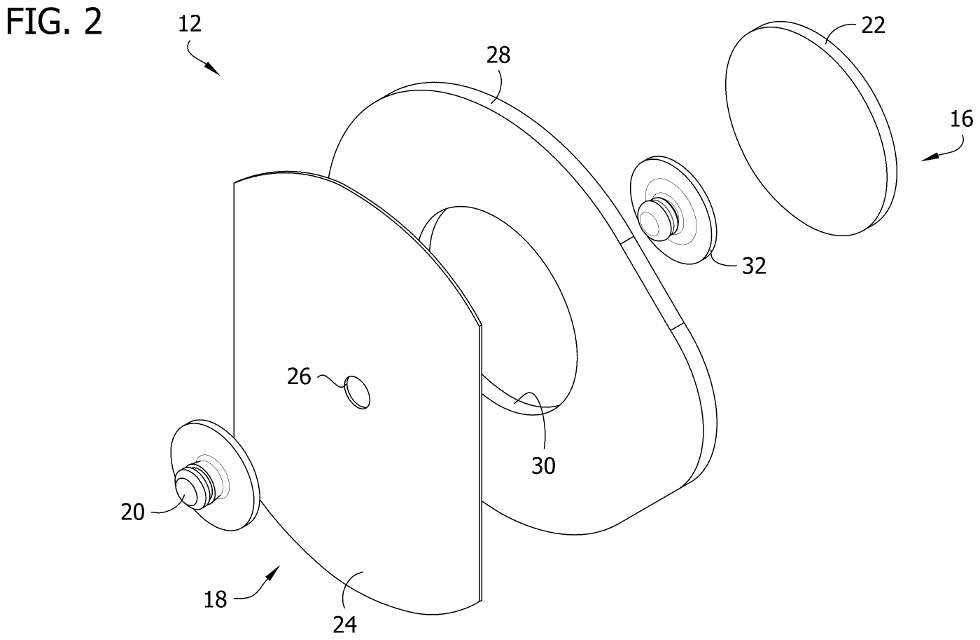

Referring to FIGS. 1 and 2 generally, each electrode assembly 12 (e.g., an ECG electrode assembly) is configured to be secured to the patient's skin at one side of the electrode assembly and connect to a dedicated connector 10 at an opposite side of the electrode assembly. The connectors 10 may be part of a lead set 15 that is connectable to a monitoring device (not shown) so that the electrode assemblies 12 can measure (or monitor) the electrical activity of the heart and transmit a signal to the monitoring device for observation by a clinician. In the illustrated embodiment, each electrode assembly 12 may include a patient contact side 16 and a connector side 18. The patient contact side 16 of the electrode assembly 12 may include biocompatible conductive gel and/or adhesive for affixing the electrode assembly to a patient's body for facilitating an appropriate electrical connection between a patient's body and the electrode assembly, as is known in the art. The connector side 18 of the electrode assembly 12 may incorporate a stud 20 for coupling the electrode assembly to the connector 10. A detailed description of the stud 20 is provided herein below.

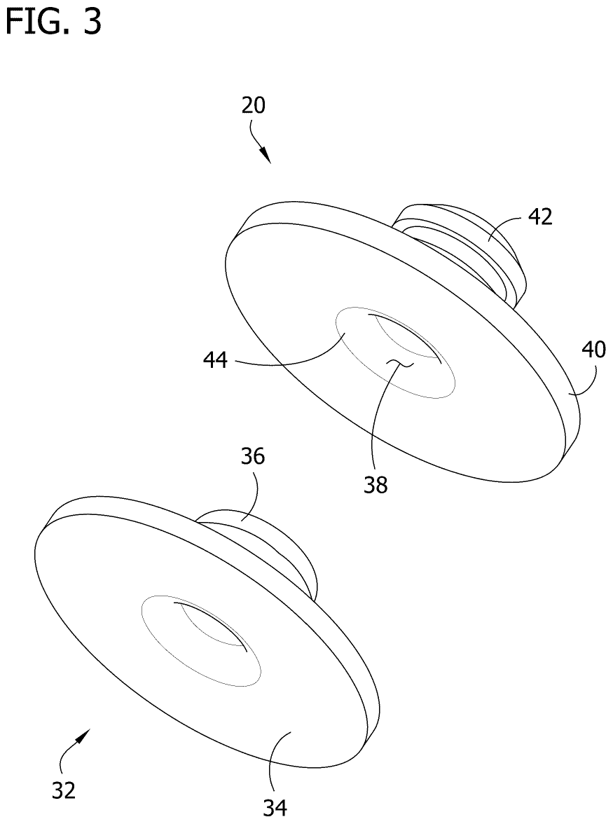

Referring to FIGS. 2 and 3, each electrode assembly 12 comprises an electrically-conductive electrode pad 22 on the patient contact side 16 for application to a surface of the patient's skin for transmitting electrical signals from the patient. In the illustrated embodiment, the electrode pad 22 comprises a disc-shaped member. As will be understood by those skilled in the art, the electrode pad 22 may be used to decrease impedance between the patient's skin and the electrode assembly 12. As such, the electrode pad 22 may have any suitable configuration, many of which are generally known in the art. In one embodiment, the electrode pad 22 comprises an electrode gel. For example, the electrode pad 22 may comprise a hydrogel.

Referring to FIG. 2, a backing layer 24 (broadly, a base) is disposed on the connector side 18 and has an opening 26 over which the stud 20 is disposed. The backing layer 24 may be configured to hold or anchor the electrical components of the electrode assembly 12. In one embodiment, the backing layer 24 may comprise a flexible film. For example, the backing layer 24 may comprise PET or TPU. However, the backing layer 24 may be formed from other materials without departing from the scope of the disclosure.

A securing layer 28 may be disposed beneath the backing layer 24 and be configured to secure the electrode assembly 12 to the patient's skin. In the illustrated embodiment, the securing layer 28 comprises a flexible ring member. Thus, the securing layer 28 may also at least partially receive the conductive member 22 within an opening 30 (e.g., a circular opening) in the securing layer. As such, the securing layer 28 and conductive member 22 may define the patient contact components of the electrode assembly 12. In the illustrated embodiment, the opening 30 has a circular shape for accommodating the disc-shaped conductive member 22. However, the opening 30 may have other shapes without departing from the scope of the disclosure. A bottom or inner side of the securing layer 28 may be treated (e.g., coated) with a biocompatible adhesive to removably adhere the electrode assembly 12 to the skin. The securing layer 28 may be formed from any suitable material. In one embodiment, the securing layer 28 comprises a foam material. For example, the securing layer 28 may comprise a PE foam. However, the securing layer 28 may be formed form other materials and have other configurations without departing from the scope of the disclosure.

The patient side 16 of the electrode assembly 12 (e.g., conductive member 22 and securing layer 28) may be temporarily covered by a release liner (not shown) to protect the patient side of the electrode assembly prior to use. The release liner may comprise a release paper or film. For example, the release liner may comprise a wax or coated plastic, such as a silicone coated polyethylene terephthalate film.

Still referring to FIGS. 2 and 3, an eyelet 32 mechanically and electrically connects the electrode pad 22 with the stud 20. The eyelet 32 may include a base portion 34 and a post 36 extending outwardly (e.g., upwardly) from the base portion. The base portion 34 is seated on and in electrically contact with an outer or upper surface of the conductive member 22 and disposed between the conductive member and the backing layer 24. The post 36 extends through the opening 26 in the backing layer 24. The post 36 is configured to be received within a passage 38 in the stud 20 and electrically and mechanically connect with the stud. For instance, a friction fit may be formed between the outer surface of the post 36 and the inner wall of the stud 20 defining the passage 38. However, the post 36 of the eyelet 32 may be secured to the stud 20 by other means without departing from the scope of the disclosure.

The eyelet 32 may be formed from any suitable material that is electrically conductive to electrically connect the electrode pad 22 with the stud 20. In one embodiment, the eyelet 32 may be formed from plastic. In one embodiment, the eyelet 32 may have a non-polarizing coating which allows the electrode assembly 12 to meet offset voltage and defibrillator recovery requirements. For example, the eyelet 32 may have an Ag/AgCl coating. Still other materials and configurations for the eyelet 32 are envisioned without departing from the scope of the disclosure. In the illustrated embodiment, the electrode assembly 12 is shown as including both the eyelet 32 and the stud 20. However, the eyelet 32 may be omitted such that the assembly 12 includes only the stud 20 without departing from the scope of the disclosure. For example, the stud 20 may be electrically and mechanically connected directly to the pad 22, or electrically and mechanically connected to the pad in other ways.

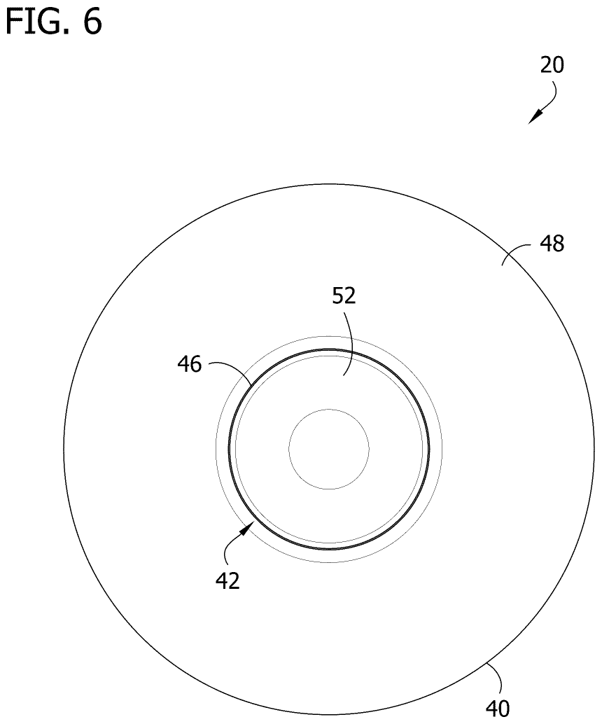

Referring to FIGS. 2-6, the stud 20 may be disposed on an upper surface of the backing layer 24 such that the stud and eyelet 32 sandwich a portion of the backing layer surrounding the opening 26 in the backing layer. The stud 20 may comprise a flange portion 40 and a retention portion 42 extending outwardly (e.g., upwardly) from the flange portion. The passage 38 extends through the flange portion 40 and into the retention portion 42 so that the post 36 on the eyelet 32 may be received into the stud 20 for a press fit connection, for example. However, it will be understood that the passage 38 may be omitted if the eyelet 32 is omitted from the electrode assembly 12. In the illustrated embodiment, the flange portion 40 comprises a ring shaped member defining an opening 44 leading into the passage 38. An outer diameter of the flange portion 40 is greater than a size of the opening 26 in the backing layer 24. As such, the flange portion 40 is sized and shaped to cover the opening 26 in the backing layer 24. However, the flange portion 40 may have other configurations without departing from the scope of the disclosure. The stud 20 may be formed from any suitable material. In one embodiment, the stud 20 comprises a metallic stud. For example, the stud 20 may be a stamped nickel plated brass. Alternatively, the stud 20 may be formed from an electrically conductive plastic material. For example, the stud 20 may comprise an injection molded carbon filled ABS. Still other materials for the stud 20 are envisioned without departing from the scope of the disclosure.

As will be explained in greater detail below, the retention portion 42 of the stud 20 does not have the bulbous shape of conventional press studs whereby an outer cross-sectional dimension of the retention portion tapers continuously from and upper portion to a lower portion such that the upper portion has a greater cross-sectional dimension than the lower portion. Rather, the configuration of the retention portion 42 is modified to improve the connection capabilities of the electrode assembly 12.

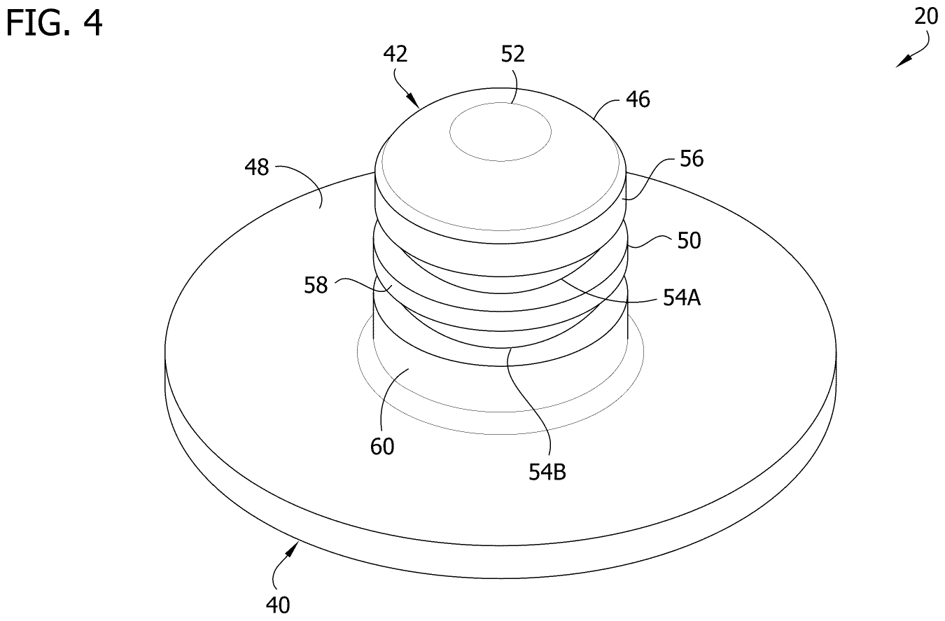

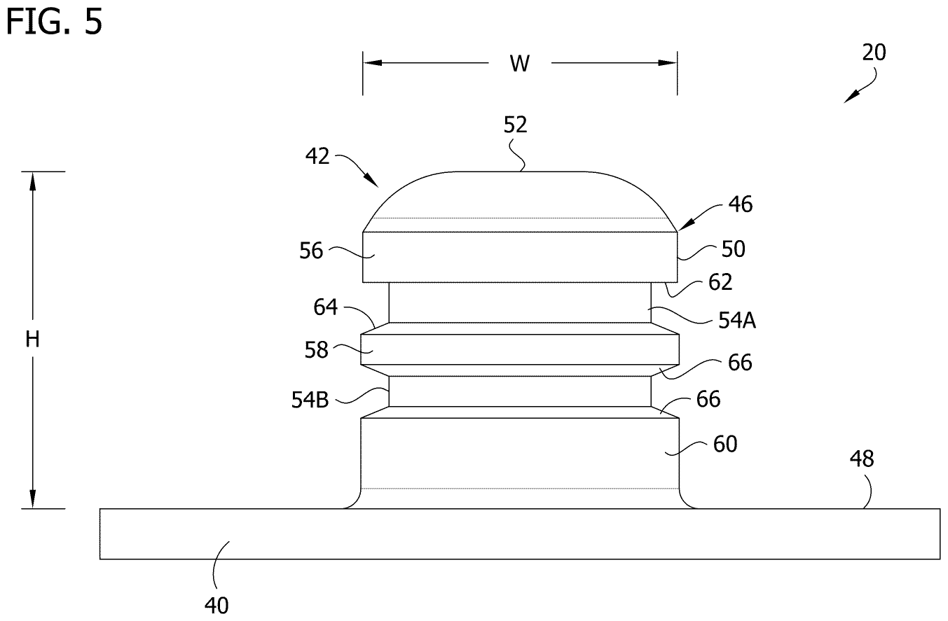

Referring to FIGS. 4-6, the retention portion 42 of the stud 20 comprises a generally cylindrical projection 46 extending from an upper surface 48 of the flange portion 40. The projection 46 includes an outer side surface 50 extending around a perimeter of the projection, and a domed upper surface 52 extending from a top of the side surface. The outer side surface 50 extends along a height H of the projection 46 and defines an outer dimension or width W of the projection (FIG. 5). The domed upper surface 52 provides a top of the projection 46 with a rounded surface for engaging certain connector types as will be discussed in greater detail below. In one embodiment, the height H of the projection 46 is between about 0.1 inches (2.54 mm) and about 0.2 inches (5.08 mm). For example, the height H may be about 0.15 inches (3.81 mm). In one embodiment, the width W (e.g., outer diameter) of the projection 46 is between about 0.1 inches (2.54 mm) and about 0.2 inches (5.08 mm). For example, the width W may be about 0.16 inches (4.064 mm). However, the projection 46 may be sized differently without departing from the scope of the disclosure.

Referring to FIGS. 4 and 5, a plurality of annular recesses 54 (broadly, retainers) are formed in the outer side surface 50 of the stud projection 46. The recesses 54 provide areas for receiving connection components on the connectors 10 for retaining the connectors to the stud 20. In one embodiment, the recesses 54 configure the stud 20 such that the stud is able to connect to different known types of ECG connectors as will be explained in greater detail below. In the illustrated embodiment, a first recess 54A is formed between a first section 56 (i.e., upper section) of the outer side surface 50 of the projection 46 and a second section 58 (i.e., middle section) of the outer side surface. A second recess 54B is formed between the second section 58 of the outer side surface 50 and a third section 60 (i.e., lower section) of the outer side surface. The first, second, and third sections 56, 58, 60 of the outer side surface 50 each define portions of the projection 46 having an outer diameter or width W that is constant along the height H of the projection. However, the first, second, and third sections 56, 58, 60 could be otherwise configured without departing from the scope of the disclosure. For example, the number of sections and recesses could be other than shown and described within the scope of the present invention. In one embodiment, a height (i.e., distance extending along the height of the projection 46) of the first recess 54A may be between about 0.02 inches (0.508 mm) and about 0.03 inches (0.762 mm). For example, the height of the first recess 54A may be about 0.023 inches (0.5842 mm). In one embodiment, a height of the second recess 54B may be between about 0.02 inches (0.508 mm) and about 0.03 inches (0.762 mm). For example, the height of the second recess 54B may be about 0.023 inches (0.5842 mm).

Referring to FIG. 5, a first transition surface 62 extends from the first section 56 of the outer side surface 50 to a bottom wall of the first recess 54A, and a second transition surface 64 extends from the second section 58 of the outer side surface to the bottom wall of the first recess. The first transition surface 62 extends generally orthogonally from the first section 56 forming a shoulder at an upper margin of the projection 46. As will be understood, an amount the first transition surface 62 extends inward from the outer side surface 50 defines a depth of the first recess 54A and also a width of the projection 46 at the first recess. Thus, in one embodiment, a depth of the first recess 54A is between about 0.02 inches (0.508 mm) and about 0.03 inches (0.762 mm). For example, a depth of the first recess 54A may be about 0.028 inches (0.7112 mm). A width (e.g., outer diameter) of the projection 46 at the first recess 54A may therefore be between about 0.1 inches (2.54 mm) and about 0.15 inches (3.81 mm). In one embodiment, the width of the projection 46 at the first recess 54A is at least about 0.1 inches (2.54 mm). For example, the width of the projection 46 at the first recess 54A may be about 0.13 inches (3.302 mm). The second transition surface 64 extends from the second section 58 at an angle toward the first transition surface 60. Thus, the second transition surface 64 may define a ramp surface for locating a connector within the first recess 54A as will be discussed in greater detail below. It will be understood, however, that the first and second transition surfaces 62, 64 could extend at other angles and be otherwise configured without departing from the scope of the disclosure.

A pair of third transition surfaces 66 extend from the second and third sections 58, 60 of the outer side surface 50, respectively, to a bottom wall of the second recess 54B. The third transition surfaces 66 extend from the second and third sections 58, 60 at an angle toward each other. Thus, the third transition surfaces 66 may individually define ramp surfaces and collectively define a funnel for locating a connector within the second recess 54B as will be discussed in greater detail below. It will be understood, however, that the third transition surfaces 66 could be otherwise configured without departing from the scope of the disclosure. In one embodiment, a depth of the second recess 54B is between about 0.02 inches (0.508 mm) and about 0.03 inches (0.762 mm). For example, a depth of the second recess 54B may be about 0.028 inches (0.7112 mm). A width of the projection 46 at the second recess 54B may therefore be between about 0.1 inches (2.54 mm) and about 0.15 inches (3.81 mm). In one embodiment, the width of the projection 46 at the second recess 54B is at least about 0.1 inches (2.54 mm). For example, the width of the projection 46 at the second recess 54B may be about 0.13 inches (3.302 mm).

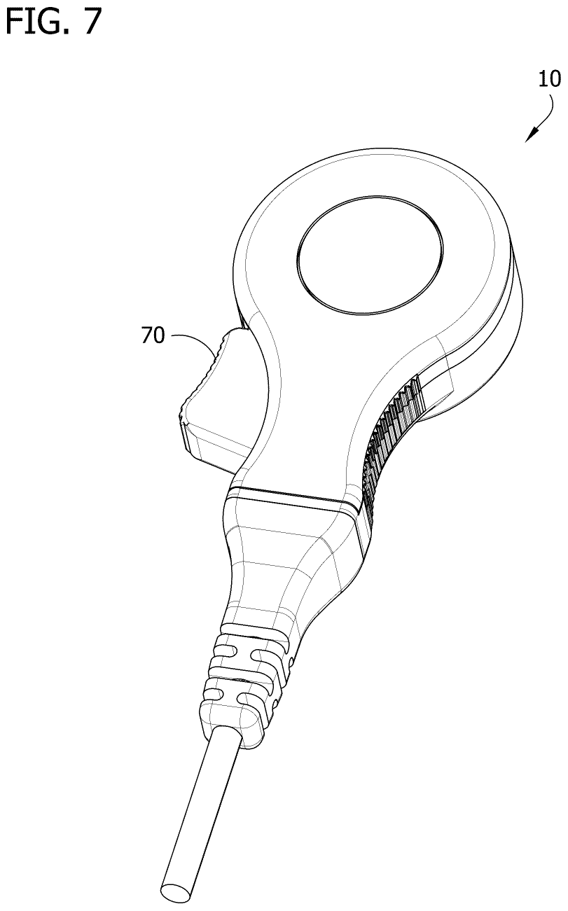

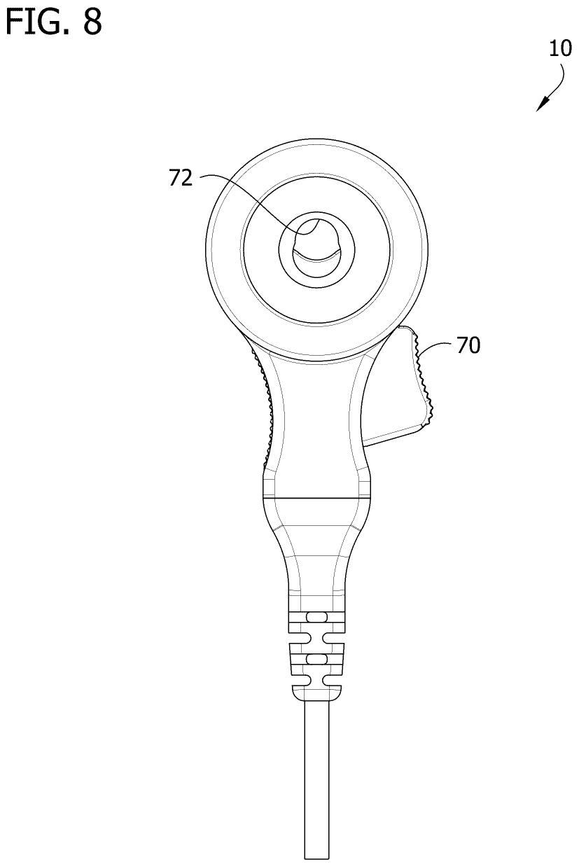

Referring to FIGS. 7 and 8, a lever-type connector 10 is disclosed for attachment (e.g., electrical connection) to the electrode assembly 12 for communication of the electrode assembly with a monitoring device. As explained in more detail below, the electrode assembly 12 may be designed to be used with other connector types without departing from the scope of the disclosure. Connection of the connector 10 to the electrode assembly 12 occurs when a lever 70 is actuated to enable an electrical contact defining a contact opening 72 (FIG. 8) to receive at least a portion of the stud 20. The lever 70 can then be released so that a biasing member (e.g., spring; not shown) moves the lever against the outer side surface 50 of the stud 20 so that the electrical contact is received in the first recess 54A, in electrical contact with the stud, and the lever retains the stud in the contact opening 72. Thus, the biased lever 70 helps to inhibit removal or electrical disconnection of the electrode assembly 12 from the connector 10. Additionally, the configuration of the stud 20 provides an enhanced attachment of the connector 10 to the electrode assembly 12. In particular, the second recess 54B provides an area on the stud 20 for receiving the electrical contact of the connector 10, such as when the connector is canted or angled relative to the stud, or another lever-type connector. Therefore, when the stud 20 is inserted into the contact opening 72 in the connector 10, the electrical contact defining the opening 72 may be positioned in registration with the second recess 54B. When the biasing member moves the lever 70 against the stud 20, the third transition surfaces 66 may serve as locators for directing the electrical contact into the second recess 54B. The gradual taper of the third transition surfaces 66 from the outer side surface 50 to the bottom of the second recess 54B may also equip the stud 20 with the necessary tolerance to accommodate lever-type connectors of various sizes and shapes. Thus, the stud 20 is configured to securely connect to different lever-type ECG connectors regardless of size or configuration. For example, the Cardinal Health™ Kendall DL™ ECG/EKG Leads Wires may be used with the disclosed electrode assembly 12.

Once the electrical contact is received in second recess 54B, the upper third transition surface 66 opposes a top of the electrical contact preventing the connector 10 from being pulled upward to detach the connector from the electrode assembly 12. Therefore, the electrode assembly 12 is configured to further resist the forces applied to the connector 10 tending to detach the connector from the electrode assembly during use. However, when it is time to disconnect the connector 10 from the electrode assembly 12, the angled upper third transition surface 66 facilitates removal once the lever 70 is actuated to disengage the electrical contact with the second recess 54B.

While the lever-type connector 10 is shown in the illustrated embodiment, the electrode assembly 12 may be used with other connector types without departing from the scope of the disclosure. For example, jaw type connectors, snap connectors, push button connectors, and “wire out of top” connectors may also securely attach to the electrode assembly 12. In the instance where a jaw type connector is used, the electrode assembly 12 is configured to engage the connector in a similar fashion to how the electrode assembly engages the lever-type connector 10 described above. In particular, the arms of the jaw type connectors may be received in either the first or second recesses 54A, 54B of the stud 20 to attach the connector to the electrode assembly 12. Suitable jaw type connectors for use with the electrode assembly 12 include the Philips Patient Cable ECG Grabbers and the GE Healthcare ECG Leadwire Grabbers. For snap connectors, the stud 20 of the electrode assembly 12 can be inserted into an opening of the connector. The domed upper surface 52 may facilitate insertion of the stud 20 by using the rounded sides of the upper surface to guide insertion of the stud. Once the stud 20 has been at least partially inserted into the connector, the first recess 54A may be positioned to receive the snap connection elements of the snap connector to attach the snap connector to the electrode assembly 12. The electrode assembly 12 provides for a more secure connection with the snap connector because the shoulder on the stud 20 formed by the first transition surface 62 opposes the snap connection elements of the snap connector preventing the connector from being pulled upward to detach the connector from the electrode assembly 12. Moreover, the second recess 54B may receive a portion of the snap connector if the snap connector is canted downward relative to the stud, so that an upper portion of the snap connector is received in the first recess 54A and a lower portion of the snap connector is received in the lower recess 54B. In one embodiment, the stud 20 is configured such that between about 2 lbs of force (8.90 N) and about 7 lbs of force (31.14 N) are require to disconnect a snap connector from the stud. Suitable snap connectors for use with the electrode assembly 12 include Cardinal Health Snap Leadwires, Nihon Kohden Direct-Connect EKG Cables, and Mindray ECG Snap Lead Wires. Accordingly, the dual retention feature of the electrode assembly 12 configures the electrode assembly to accommodate any type of conventional ECG connector. As such, the electrode assembly 12 provides for universal attachment to all ECG connectors.

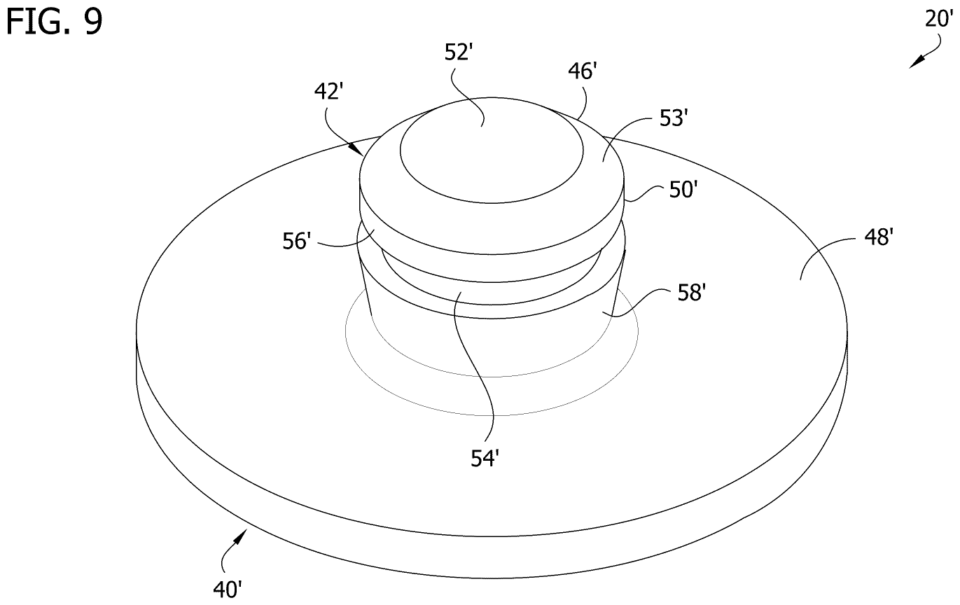

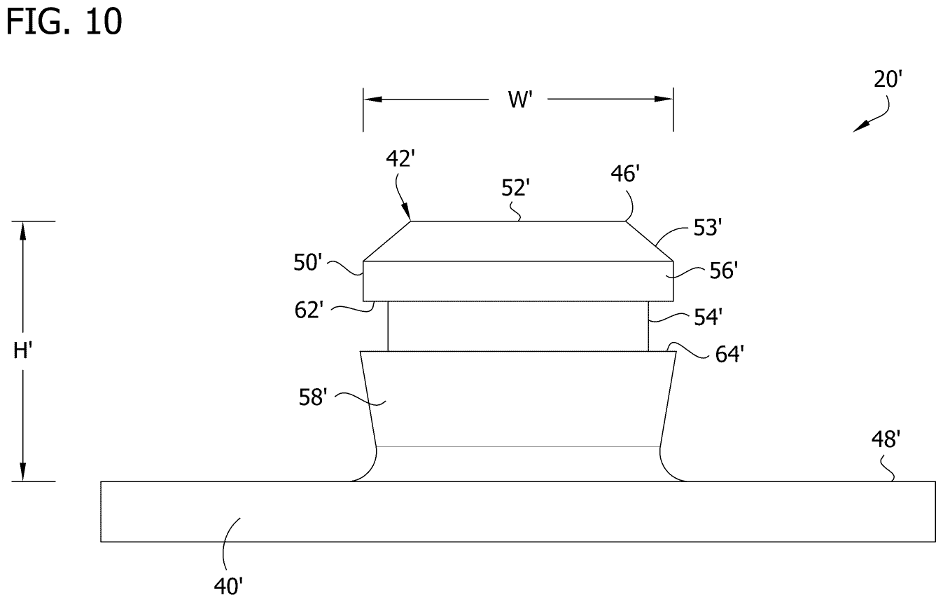

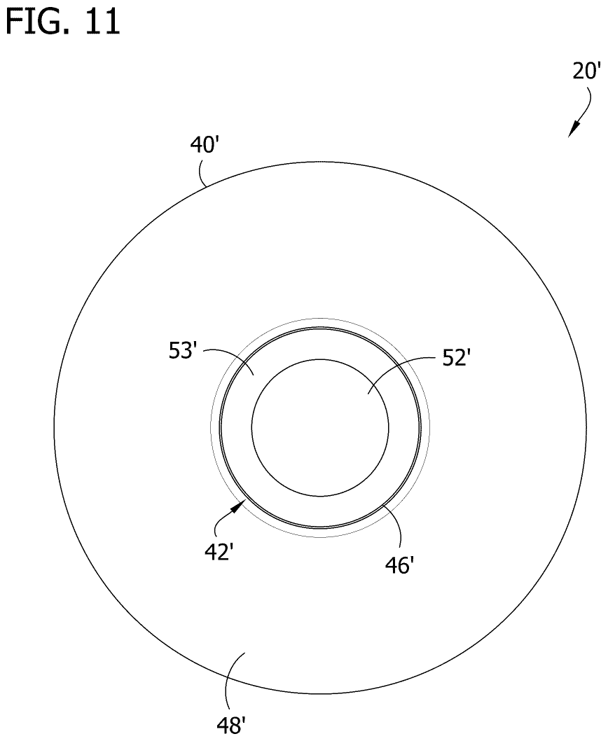

Referring to FIGS. 9-11, a stud of another embodiment is generally indicated at 20′. The stud 20′ comprises a flange portion 40′ and a retention portion 42′ extending outwardly (e.g., upwardly) from the flange portion. The retention portion 42′ of the stud 20′ comprise a generally cylindrical projection 46′ extending from an upper surface 48′ of the flange portion 40′. The projection 46′ includes an outer side surface 50′ extending around a perimeter of the projection, and a flat upper surface 52′ surrounded by a downwardly angled side surface 53′. The outer side surface 50′ extends along a height H′ of the projection 46′ and defines an outer dimension or width W′ (e.g., outer diameter) of the projection (FIG. 10). The angled side surface 53′ provides a top of the projection 46′ with an angled surface for engaging certain connector types. In one embodiment, the height H′ of the projection 46′ is between about 0.1 inches (2.54 mm) and about 0.2 inches (5.08 mm). For example, the height H′ may be about 0.13 inches (3.302 mm). In one embodiment, the width W′ of the projection 46′ is between about 0.1 inches (2.54 mm) and about 0.2 inches (5.08 mm). For example, the width W′ may be about 0.16 inches (4.064 mm). However, the projection 46′ may be sized differently without departing from the scope of the disclosure.

Referring to FIGS. 9 and 10, an annular recess 54′ (broadly, a retainer) is formed in the outer side surface 50′ of the stud projection 46′. The recess 54′ and side surface 50′ provide areas for receiving connection components on a connector for retaining the connectors to the stud 20′. In one embodiment, the recess 54′ and side surface 50′ configure the stud 20′ such that the stud is able to connect to any type of ECG connector as will be explained in greater detail below. In the illustrated embodiment, the recess 54′ is formed between a first section 56′ (i.e., upper section) of the outer side surface 50′ of the projection 46′ and a second section 58′ (i.e., lower section) of the outer side surface. The first section 56′ of the outer side surface 50′ defines a portion of the projection 46′ having an outer diameter or width W′ that is constant along the height H′ of the projection. The second section 58′ of the outer side surface 50′ defines a portion of the projection 46′ having an outer diameter of width W′ that tapers from an upper margin to a lower margin. However, the first and second sections 56′, 58′ could be otherwise configured without departing from the scope of the disclosure. The second section 58′ may be broadly considered a retainer as will be discussed in greater detail below. In one embodiment, a height (i.e., distance extending along the height of the projection 46′) of the recess 54′ may be between about 0.02 inches (0.508 mm) and about 0.03 inches (0.762 mm). For example, the height of the recess 54′ may be about 0.025 (0.635 mm). In one embodiment, a height of the second section 58′ may be between about 0.06 inches (1.524 mm) and about 0.07 inches (1.778 mm). For example, the height of the second section 58′ may be about 0.065 inches (1.651 mm).

Referring to FIG. 10, a first transition surface 62′ extends from the first section 56′ of the outer side surface 50′ to a bottom wall of the recess 54′, and a second transition surface 64′ extends from the second section 58′ of the outer side surface to the bottom wall of the recess. The first and second transition surfaces 62′, 64′ extend generally orthogonally from the first and second sections 56′, 58′, respectively, forming a top shoulder and a bottom ledge on the projection 46′. As will be understood, an amount the first and second transition surfaces 62′, 64′ extend inward from the outer side surface 50′ defines a depth of the recess 54′ and also a width of the projection 46′ at the recess. Thus, in one embodiment, a depth of the recess 54′ is between about 0.02 inches (0.508 mm) and about 0.03 inches (0.762 mm). For example, a depth of the recess 54′ may be about 0.028 inches (0.7112 mm). A width of the projection 46′ at the recess 54′ may therefore be between about 0.1 inches (2.54 mm) and about 0.15 inches (3.81 mm). In one embodiment, the width of the projection 46′ at the recess 54′ is at least about 0.1 inches (2.54 mm). For example, the width of the projection 46′ at the recess 54′ may be about 0.13 inches (3.302 mm). It will be understood, however, that the first and second transition surfaces 62′, 64′ could extend at other angles and be otherwise configured without departing from the scope of the disclosure. For instance, the first and second transition surfaces 62′, 64′ could be angled toward each other.

Referring to FIGS. 7, 8, and 10, connection of a lever-type connector 10 to an electrode assembly 12 including stud 20′ occurs when the lever 70 is actuated to allow the contact opening 72 (FIG. 8) in the connector to receive at least a portion of the stud 20′. The lever 70 can then be released so that a biasing member (not shown) moves the lever against the outer side surface 50′ of the stud 20′ to retain the stud in the contact opening with the stud in contact with the electrical contact of the connector. Thus, the biased lever 70 helps to inhibit removal or electrical disconnection of the electrode assembly 12 from the connector 10. Additionally, the configuration of the stud 20′ provides an enhanced attachment of the connector 10 to the electrode assembly 12. In particular, the second section 58′ of the outer side surface 50′ provides an area on the stud 20′ for contacting the electrical contact of the connector 10. Also, when the stud 20′ is inserted into the contact opening 72 in the connector 10, the lever 70 may be positioned in registration with the second section 58′. When the biasing member moves the lever 70 against the stud 20′, the lever and the electrical contact will come into contact with the second section 58′. The tapered profile of the second section 58′ may serve as a locator for directing the lever 70 and the electrical contact onto the second section 58′. The height of the second section 58′ may also equip the stud 20′ with the necessary tolerance to accommodate lever-type connectors of various sizes and shapes.

Once the lever 70 is moved into engagement with the second section 58′ of the outer side surface 50′, the upper portion of the second section 58′ generally prevent the connector 10 from being pulled upward to detach the connector from the electrode assembly 12. Therefore, the electrode assembly 12 is configured to further resist the forces applied to the connector 10 tending to detach the connector from the electrode assembly during use. However, when it is time to disconnect the connector 10 from the electrode assembly 12, the angled second section 58′ facilitates removal after the lever 70 is actuated to disengage the lever with the outer side surface 50′. Moreover, the second section 58′ may receive a portion of the connector 10′ if the connector is canted downward relative to the stud, so that an upper portion of the snap connector is received in the recess 54 and a lower portion of the snap connector engages the second section. Thus, the stud 20′ is configured to securely connect to different lever-type ECG connectors regardless of size or configuration. For example, the Cardinal Health™ Kendall DL™ ECG/EKG Leads Wires may be used with the disclosed electrode assembly 12.

As previously discussed, the electrode assembly 12 may be used with other connector types without departing from the scope of the disclosure. For example, jaw type connectors, snap connectors, push button connectors, and “wire out of top” connectors may also securely attach to the electrode assembly 12. In the instance where a jaw type connector is used, the electrode assembly 12 including stud 20′ is configured to engage the connector in a similar fashion to how the electrode assembly engages the lever-type connector described above. In particular, the arms of the jaw type connectors may be received in the recess 54′ and/or around the second section 58′ of the outer side surface 50′ of the stud 20′ to attach the connector to the electrode assembly 12. Suitable jaw type connectors for use with the electrode assembly 12 include the Philips Patient Cable ECG Grabbers and the GE Healthcare ECG Leadwire Grabbers. For snap connectors, the stud 20′ of the electrode assembly 12 can be inserted into an opening of the connector. The angled sides surrounding the flat upper surface 52′ may facilitate insertion of the stud 20′ by using the angled sides of the upper surface to guide insertion of the stud. Once the stud 20′ has been at least partially inserted into the connector, the recess 54′ may be positioned to receive the snap connection elements of the snap connector to attach the snap connector to the electrode assembly 12. The electrode assembly 12 provides for a more secure connection with the snap connector because the shoulder on the stud 20′ formed by the first transition surface 62′ opposes the snap connection elements of the snap connector preventing the connector from being pulled upward to detach the connector from the electrode assembly 12. Moreover, the second section 58′ may receive a portion of the snap connector if the snap connector is canted downward relative to the stud, so that an upper portion of the snap connector is received in the recess 54′ and a lower portion of the snap connector is received in the second section 58′. Suitable snap connectors for use with the electrode assembly 12 include Cardinal Health Snap Leadwires, Nihon Kohden Direct-Connect EKG Cables, and Mindray ECG Snap Lead Wires. Accordingly, the dual retention feature of the electrode assembly 12 including stud 20′ also configures the electrode assembly to accommodate any type of conventional ECG connector. As such, the electrode assembly 12 provides for universal attachment to all ECG connectors.

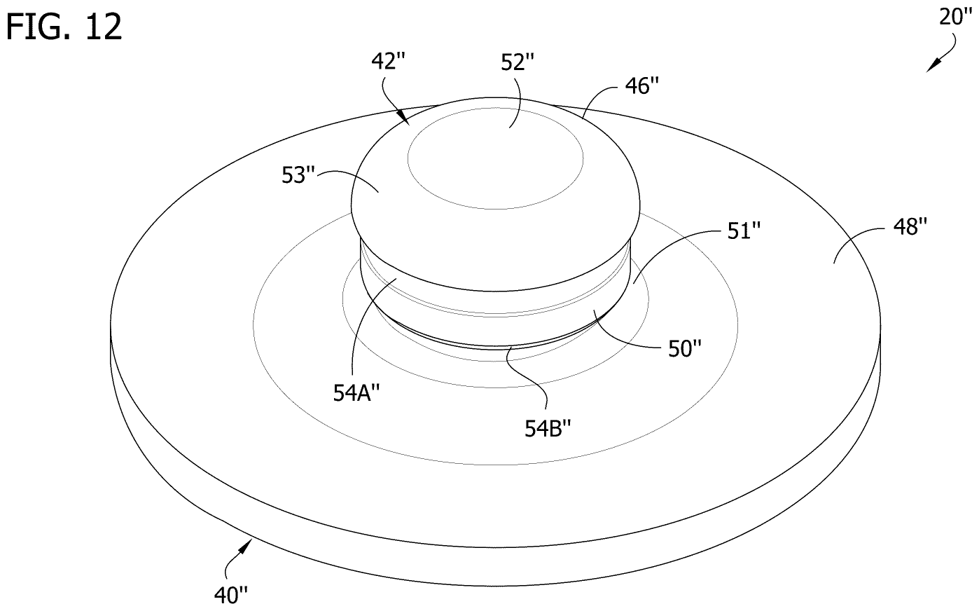

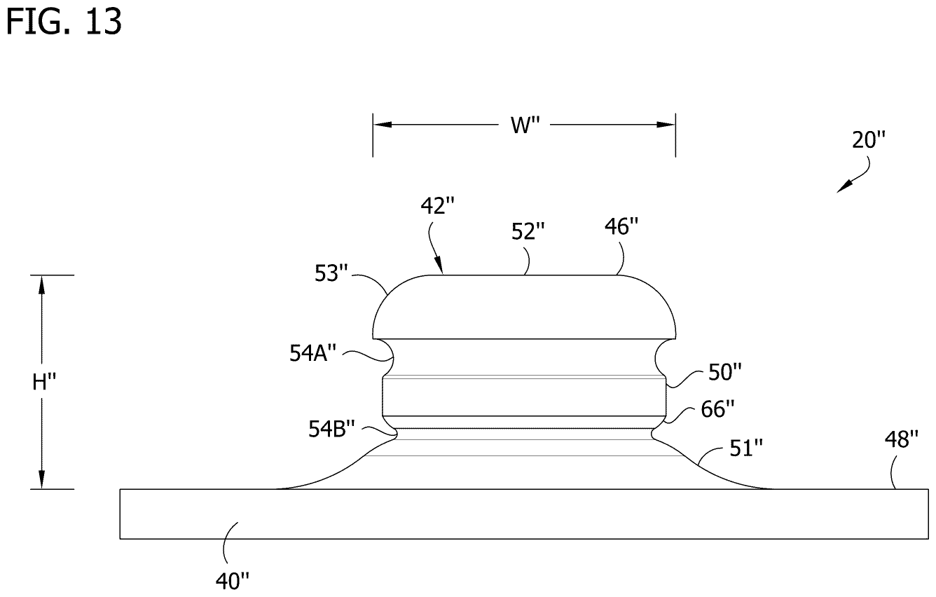

Referring to FIGS. 12 and 13, a stud of another embodiment is generally indicated at 20″. The stud 20″ comprises a flange portion 40″ and a retention portion 42″ extending outwardly (e.g., upwardly) from the flange portion. The retention portion 42″ of the stud 20″ comprises a partially cylindrical projection 46″ extending from an upper surface 48″ of the flange portion 40″. The projection 46″ includes an outer side surface 50″ extending around a perimeter of the projection, an inclined surface 51″ extending between the upper surface 48″ and the outer side surface 50″, and a flat upper surface 52″ surrounded by a downwardly curved side surface 53″ extending between the flat upper surface and the outer side surface. The outer side surface 50″ extends along a height H″ of the projection 46″ and defines an outer dimension or width W″ of the cylindrical portion of the projection (FIG. 13). The curved side surface 53″ provides a top of the projection 46″ with a curved surface for engaging certain connector types. In one embodiment, the height H″ of the projection 46″ is between about 0.1 inches (2.54 mm) and about 0.2 inches (5.08 mm). For example, the height H″ may be about 0.1 inches (2.54 mm). In one embodiment, the width W″ of the projection 46″ is between about 0.1 inches (2.54 mm) and about 0.2 inches (5.08 mm). For example, the width W″ may be about 0.15 inches (3.81 mm). However, the projection 46″ may be sized differently without departing from the scope of the disclosure.

A plurality of annular recesses 54″ (broadly, retainers) are formed in the side of the stud projection 46″. The recesses 54″ provide areas for receiving connection components on the connectors 10 for retaining the connectors to the stud 20″. In one embodiment, the recesses 54″ configure the stud 20″ such that the stud is able to connect to different known types of ECG connectors as will be explained in greater detail below. In the illustrated embodiment, a first recess 54A″ is formed between the curved side surface 53″ of the projection 46″ and the outer side surface 50″. A second recess 54B″ is formed between the outer side surface 50″ and the inclined surface 51″. The outer side surface 50″ has an outer diameter or width W″ that is constant along the height H″ of the projection. However, the outer side surface 50″ could be otherwise configured without departing from the scope of the disclosure. In one embodiment, a height (i.e., distance extending along the height of the projection 46″) of the first recess 54A″ may be between about 0.01 inches (0.254 mm) and about 0.02 inches (0.508 mm). For example, the height of the first recess 54A″ may be about 0.015 inches (0.381 mm). In one embodiment, a height of the second recess 54B″ may be between about 0.01 inches (0.254 mm) and about 0.02 inches (0.508 mm). For example, the height of the second recess 54B″ may be about 0.01 inches (0.254 mm).

Referring to FIG. 13, the first recess 54A″ is defined by a concave wall disposed above the outer side surface 50″. Thus, the first recess 54A″ is disposed in a top half of the projection 46″. In one embodiment, a depth of the first recess 54A″ is between about 0.02 inches (0.508 mm) and about 0.03 inches (0.762 mm). For example, a depth of the first recess 54A″ may be about 0.025 inches (0.635 mm). A width of the projection 46″ at the first recess 54A″ may therefore be between about 0.1 inches (2.54 mm) and about 0.15 inches (3.81 mm). In one embodiment, the width of the projection 46″ at the first recess 54A″ is at least about 0.1 inches (2.54 mm). For example, the width of the projection 46″ at the first recess 54A″ may be about 0.13 inches (3.302 mm). The second recess 54B″ is defined by a concave wall disposed below the outer side surface 50″. In the illustrated embodiment, the second recess 54B″ defines a bottom of the cylindrical portion of the projection 46″. Thus, the second recess 54B″ is disposed in a bottom half of the projection 46″. A transition surface 66″ extends from the outer side surface 50″ to the concave wall defining the second recess 54B″. In the illustrated embodiment, the transition surface 66″ comprises a convex wall oriented so as to extend into the projection 46′ as the transition surface extends from top to bottom in FIG. 13. In one embodiment, a depth of the second recess 54B″ is between about 0.02 inches (0.508 mm) and about 0.03 inches (0.762 mm). For example, a depth of the second recess 54B″ may be about 0.025 inches (0.635 mm). A width of the projection 46″ at the second recess 54B″ may therefore be between about 0.1 inches (2.54 mm) and about 0.15 inches (3.81 mm). In one embodiment, the width of the projection 46″ at the second recess 54B″ is at least about 0.1 inches (2.54 mm). For example, the width of the projection 46″ at the second recess 54B″ may be about 0.13 inches (3.302 mm). In one embodiment, the outer side surface 50″ may include the inclined surface 51″ and the curved side surface 53″.

Referring to FIGS. 7, 8, and 13, connection of a lever-type connector 10 to an electrode assembly 12 including stud 20″ occurs when the lever 70 is actuated to allow the contact opening 72 (FIG. 8) in the connector to receive at least a portion of the stud 20″. The lever 70 can then be released so that a biasing member (not shown) moves the lever against the outer side surface 50″ of the stud 20″ so that the electrical contact is received in the first recess 54A″, in electrical contact with the stud, and the lever retains the stud in the contact opening 72. Thus, the biased lever 70 helps to inhibit removal or electrical disconnection of the electrode assembly 12 from the connector 10. Additionally, the configuration of the stud 20″ provides an enhanced attachment of the connector 10 to the electrode assembly 12. In particular, the second recess 54B″ provides an area on the stud 20″ for contacting the electrical contact of the connector 10, such as when the connector is canted or angled relative to the stud, or another lever-type connector. Therefore, when the stud 20″ is received in the contact opening 72 in the connector 10, the electrical contact may be positioned in registration with the second recess 54B″. When the biasing member moves the lever 70 against the stud 20″, the electrical contact will come into contact with the bottom of the second recess 54B″. The concave shape of the second recess 54B″ may serve as a locator for directing the electrical contact into the second recess. The inclined surface 51″ may also function to locate the lever 70 into the second recess 54B″. Also, the gradual taper of the transition surfaces 66″ from the outer side surface 50″ to the second recess 54B″ may equip the stud 20″ with the necessary tolerance to accommodate lever-type connectors of various sizes and shapes. Thus, the stud 20″ is configured to securely connect to different=lever-type ECG connectors regardless of size or configuration. For example, the Cardinal Health™ Kendall DL™ ECG/EKG Leads Wires may be used with the disclosed electrode assembly 12.

Once the electrical contact is received in the second recess 54B″, the transition surface 66″ opposes a top of the electrical contact preventing the connector 10 from being pulled upward to detach the connector from the electrode assembly 12. Therefore, the electrode assembly 12 is configured to further resist the forces applied to the connector 10 tending to detach the connector from the electrode assembly during use. However, when it is time to disconnect the connector 10 from the electrode assembly 12, the curved transition surface 66″ facilitates removal once the lever 70 is actuated to disengage the electrical contact with the second recess 54B″.

While the lever-type connector 10 is shown in the illustrated embodiment, the electrode assembly 12 incorporating stud 20″ may be used with other connector types without departing from the scope of the disclosure. For example, jaw type connectors, snap connectors, push button connectors, and “wire out of top” connectors may also securely attach to the electrode assembly 12. In the instance where a jaw type connector is used, the electrode assembly 12 is configured to engage the connector in a similar fashion to how the electrode assembly engages the lever-type connector 10 described above. In particular, the arms of the jaw type connectors may be received in either the first or second recesses 54A″, 54B″ of the stud 20″ to attach the connector to the electrode assembly 12. Suitable jaw type connectors for use with the electrode assembly 12 include the Philips Patient Cable ECG Grabbers and the GE Healthcare ECG Leadwire Grabbers. For snap connectors, the stud 20″ of the electrode assembly 12 can be inserted into an opening of the connector. The curved side surface 53″ may facilitate insertion of the stud 20″ by using the rounded sides to guide insertion of the stud, and wedging apart the jaws of the connector. Once the stud 20″ has been at least partially inserted into the connector, the first recess 54A″ may be positioned to receive the snap connection elements of the snap connector to attach the snap connector to the electrode assembly 12. The electrode assembly 12 provides for a more secure connection with the snap connector because a shoulder on the stud 20″ formed by the concave surface defining the first recess 54″ opposes the snap connection elements of the snap connector preventing the connector from being pulled upward to detach the connector from the electrode assembly 12. Moreover, the second recess 54B″ may receive a portion of the snap connector if the snap connector is canted downward relative to the stud, so that an upper portion of the snap connector is received in the first recess 54A″ and a lower portion of the snap connector is received in the lower recess 54B″. In one embodiment, the stud 20″ is configured such that between about 2 and about 7 lbs of force (31.14 N) are require to disconnect a snap connector from the stud. Suitable snap connectors for use with the electrode assembly 12 include Cardinal Health Snap Leadwires, Nihon Kohden Direct-Connect EKG Cables, and Mindray ECG Snap Lead Wires. Accordingly, the dual retention feature of the electrode assembly 12 configures the electrode assembly to accommodate any type of conventional ECG connector. As such, the electrode assembly 12 provides for universal attachment to all ECG connectors.

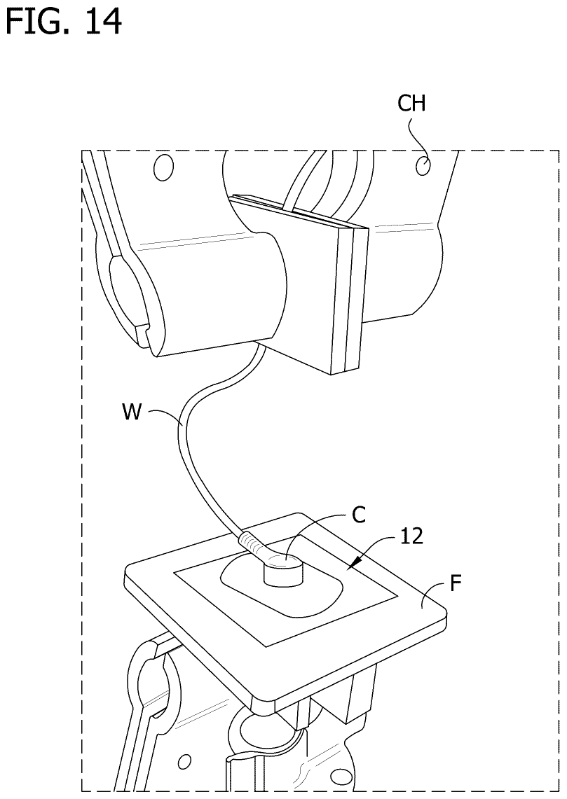

Referring to FIG. 14, a study was conducted to determine retention force readings for an electrode assembly including the stud 20″. Snap connectors C were attached to an electrode assembly 12 mounted on a lower fixture F. Lead wires W of the connectors C were secured to an Instron machine. The Instron machine was then operated to exert a pulling force on the lead wire W by moving a crosshead CH upward away from the lower fixture F. The crosshead CH was moved away from the fixture F until the connector C became detached from the electrode assembly 12. The Instron machine detected a force of at least about 2 lbs. (8.90 N) to detach the connector C from the electrode assembly 12. On average, a removal force of about 5 lbs (22.24 N). was required to detach the connector C from the electrode assembly 12. This removal force was significantly greater than the measured removal forces for conventional electrode assemblies incorporating standard bulbous studs. For example, conventional electrode assemblies have a measured removal force of about ½ lb (2.224 N). Thus, the configuration of the stud 20″ provides for a more secure attachment to standard connectors than conventional studs.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions without departing from the scope of the invention, it is intended that all matter contained in the above description and shown in the accompanying drawings shall be interpreted as illustrative and not in a limiting sense.

What is claimed is:

1. An electrode assembly comprising:
electrode pad including a patient contact side for engaging skin of a patient and a connector side for attaching a connector to the electrode pad; and
a stud mounted on the connector side of the electrode pad for directly engaging the connector to retain the connector to the electrode pad, the stud comprising a first retainer for engaging and retaining a first type of connector to the stud and a second retainer for engaging and retaining a second type of connector to the stud, the first and second types of connectors having different connection methods.

2. The assembly of claim 1, wherein the first retainer comprises a recess in an outer side surface of the stud.

3. The assembly of claim 2, wherein the second retainer comprises a second recess in the outer side surface of the stud.

4. The assembly of claim 2, wherein the second retainer comprises a section of the outer side surface of the stud spaced away from the recess.

5. The assembly of claim 1, wherein the stud comprises a generally cylindrical projection extending outwardly from the electrode pad.

6. The assembly of claim 5, wherein the projection comprises a generally cylindrical outer side surface and one of a domed upper surface or angled surfaces extending from a top of the outer side surface.

7. The assembly of claim 6, wherein the outer side surface includes a first section disposed above the first retainer and a second section disposed below the first retainer.

8. The assembly of claim 7, wherein the first retainer comprises a recess in the outer side surface.

9. The assembly of claim 7, wherein the first section has an outer dimension that is constant along a height of the projection.

10. The assembly of claim 7, wherein the second section has an outer dimension that is constant along the height of the projection.

11. The assembly of claim 7, wherein the outer side surface includes a third section disposed below the second retainer.

12. The assembly of claim 11, wherein the second retainer comprises a recess in the outer side surface.

13. The assembly of claim 7, wherein the second section defines the second retainer.

14. The assembly of claim 5, wherein the stud is free of a bulbous shape where an outer cross-sectional dimension of the stud tapers continuously from an upper portion to a lower portion such that the upper portion has a greater cross-sectional dimension than the lower portion.

15. A stud for use in an electrode assembly comprising:
a projection configured for directly engaging a connector to retain the connector to the stud, the projection comprising a first retainer for engaging and retaining a first type of connector to the stud and a second retainer for engaging and retaining a second type of connector to the stud, the first and second types of connectors having different connection methods.

16. The stud of claim 15, wherein the first retainer comprises a recess in an outer side surface of the projection.

17. The stud of claim 16, wherein the second retainer comprises a second recess in the outer side surface of the projection.

18. The stud of claim 16, wherein the second retainer comprises a section of the outer side surface of the stud spaced away from the recess.

19. The stud of claim 15, wherein the projection comprises a generally cylindrical outer side surface and one of a domed upper surface or an angled surface extending from a top of the outer side surface.

20. An electrode assembly comprising:
electrode pad including a patient contact side for engaging skin of a patient and a connector side for attaching a snap-type connector to the electrode pad; and
a stud mounted on the connector side of the electrode pad for directly engaging the snap-type connector to retain the snap-type connector to the electrode pad, the stud comprising a retainer for engaging and retaining the snap-type connector to the stud, a removal force of at least 2 lbs (8.90 N) being required for detaching the snap-type connector from the electrode assembly.
